(19) United States
(12) Patent Application Publication
(10) Pub. No.: US 2024/0316800 A1
(43) Pub. Date: Sep. 26, 2024

(54) FORCEPS UNIT AND FORCEPS APPARATUS

(71) Applicant: KOREA INSTITUTE OF SCIENCE AND TECHNOLOGY, Seoul (KR)

(72) Inventors: Yongseok IHN, Seoul (KR); Junwon LEE, Seoul (KR); Sungwook YANG, Seoul (KR); Donghyun HWANG, Seoul (KR)

(73) Assignee: KOREA INSTITUTE OF SCIENCE AND TECHNOLOGY, Seoul (KR)

(21) Appl. No.: 18/468,949

(22) Filed: Sep. 18, 2023

(30) Foreign Application Priority Data
Mar. 24, 2023 (KR) 10-2023-0038454

(51) Int. Cl. B25J 19/02; A61B 34/30

(52) U.S. Cl. CPC B25J 19/021; A61B 34/30; A61B 2034/2061

(57) ABSTRACT

A forceps unit and a forceps apparatus are provided. The forceps unit according to an aspect of the present disclosure may comprise a base portion connected to a motor; a grasping portion disposed in front of the base portion and grasping a surgical instrument; a connecting portion connecting the base portion and the grasping portion; and a plurality of optical fibers, wherein some of the plurality of optical fibers are fixed to the grasping portion and others of the plurality of optical fibers are fixed to the base portion, wherein the plurality of optical fibers may include a first optical fiber disposed in a central region of the grasping portion and second to fifth optical fibers radially disposed with respect to the first optical fiber, and the first optical fiber may protrude further forward than the second to fifth optical fibers.

### CROSS-REFERENCE TO RELATED APPLICATION

This application claims priority to and the benefit of Korean Patent Application No. 10-2023-0038454 filed in the Korean Intellectual Property Office on Mar. 24, 2023, the entire contents of which are incorporated herein by reference.

### BACKGROUND OF THE INVENTION

### Field of the Invention

The present disclosure relates to a forceps unit and a forceps apparatus for grasping surgical instruments.

### Description of the Related Art

In robotically-assisted surgery, the surgeon typically operates a master controller to control the movement of surgical instruments at the surgical site from a location remote from the patient (e.g. across from an operating room, another room, or a completely different building).

The master controller usually includes one or more manual input apparatus such as a small handheld wrist gimbal, joystick, exoskeleton glove, handpiece, etc.

The manual input apparatus is operably connected by a servo motor organically integrating surgical instruments and their location and direction in the surgical site through the controller.

The servo motor is a part of a surgical manipulation apparatus including a plurality of joints connected to each other and link apparatus to support and control the surgical instruments that are usually introduced directly into an open surgical site or through a trocar sleeve inserted through an incision into a body cavity, such as the patient's abdomen.

Depending on the surgical procedure, various surgical instruments such as a tissue grasper, a needle driver, and an electrosurgical cautery probe may be used to perform various functions, for example, the function of retracting tissue, the function of picking up or inserting a needle, the function of suturing, the function of grasping blood vessels, or the function of dissecting, the function of cauterizing or coagulating tissue. During the surgical procedure, the surgeon may use a variety of surgical instruments.

In the case of general robotic surgery, doctors mostly rely on their eyesight to sense the force applied to the surgical instruments. However, in the case of ultra-micro surgery, the force generated during the operation, such as the moment the needle penetrates the tissue, is so small that a person cannot feel it, and the minute difference in force may affect tissue damage or surgical results.

Accordingly, there is a demand for the development of a forceps apparatus capable of accurately and precisely measuring minute forces in the ultra-micro surgery.

### SUMMARY OF THE INVENTION

A problem to be solved by the present disclosure is to provide a forceps unit and a forceps apparatus capable of precisely measuring 5-axis forces by compensating for the effect of temperature change.

A forceps unit according to one aspect of the present disclosure for achieving the above object includes a base portion connected to a motor; a grasping portion disposed in front of the base portion and grasping a surgical instrument; a connecting portion connecting the base portion and the grasping portion; and a plurality of optical fibers, wherein some of the plurality of optical fibers are fixed to the grasping portion and others of the plurality of optical fibers are fixed to the base portion.

In this case, the plurality of optical fibers may include a first optical fiber disposed in a central region of the grasping portion and second to fifth optical fibers radially disposed with respect to the first optical fiber, and the first optical fiber may protrude further forward than the second to fifth optical fibers.

Through this, since the 5-axis forces can be measured by compensating for the effect of temperature change, the force applied by the surgical instrument can be accurately and precisely measured.

In addition, the first optical fiber may include a first fixing portion fixed to the grasping portion, a second fixing portion fixed to the base portion, a first brag disposed between the first fixing portion and the second fixing portion, and a second brag disposed in front of the first fixing portion.

In addition, the second optical fiber may include a third fixing portion fixed to the grasping portion, a fourth fixing portion fixed to the base portion, and a third brag disposed between the third fixing portion and the fourth fixing portion, the third optical fiber may include a fifth fixing portion fixed to the grasping portion, a sixth fixing portion fixed to the base portion, and a fourth brag disposed between the fifth fixing portion and the sixth fixing portion, the fourth optical fiber may include a seventh fixing portion fixed to the grasping portion, an eighth fixing portion fixed to the base portion, and a fifth brag disposed between the seventh fixing portion and the eighth fixing portion, and the fifth optical fiber may include a ninth fixing portion fixed to the grasping portion, a tenth fixing portion fixed to the base portion, and a sixth brag disposed between the ninth fixing portion and the tenth fixing portion.

In addition, the first brag and the third to sixth brags may be disposed on the same plane.

In addition, a back end of the first fixing portion, a back end of the third fixing portion, a back end of the fifth fixing portion, a back end of the seventh fixing portion, and a back end of the ninth fixing portion may be disposed on the same plane, and a front end of the second fixing portion, a front end of the fourth fixing portion, a front end of the sixth fixing portion, a front end of the eighth fixing portion, and a front end of the tenth fixing portion may be disposed on the same plane.

In addition, the first to sixth brags satisfy the following Equation,

$$\\ [ \begin{matrix}Δ {λ}_{L} - Δ {λ}_{R} \\ Δ {λ}_{B} - Δ {λ}_{T} \\ {\sum }_{j = B , T , C , L , R} Δ {λ}_{j} \\ Δ {λ}_{Temp}\end{matrix} ] = [ \begin{matrix}{S}_{11} & 0 & 0 & {S}_{14} \\ 0 & {S}_{22} & 0 & {S}_{24} \\ 0 & 0 & {S}_{33} & {S}_{34} \\ 0 & 0 & 0 & {S}_{44}\end{matrix} ] \cdot [ \begin{matrix}{F}_{x} \\ {F}_{y} \\ {F}_{z} \\ Δ T\end{matrix} ]$$

(where, λ_{C} is a change in length of the first brag, λ_{Temp} is a change in length of the second brag, λ_{R} is a change in length of the third brag, λ_{L} is a change in length of the fourth brag, λ_{T} is a change in length of the fifth brag, λ_{B} is a change in length of the sixth brag).

In addition, the second optical fiber may be disposed symmetrically with the third optical fiber with respect to the first optical fiber, the fourth optical fiber may be disposed symmetrically with the fifth optical fiber with respect to the first optical fiber, and intervals between the second to fifth optical fibers may be the same.

In addition, the front end of the first optical fiber may be a free end.

In addition, the base portion may include first to fifth through holes respectively penetrated by the first to fifth optical fibers and extending in a horizontal direction, the first through hole may be penetrated by the first optical fiber, the second through hole may be disposed on the right side of the first through hole, the third through hole may be disposed on the left side of the first through hole, the fourth through hole may be disposed above the first through hole, the fifth through hole may be disposed below the first through hole, the second to fourth through holes may extend downward from an upper surface of the base portion, and the first through hole may be connected to the second through hole or the third through hole.

In addition, the grasping portion may include sixth to ninth through holes respectively penetrated by the first to fourth optical fibers, the sixth through hole may be penetrated by the first optical fiber, the seventh through hole may be disposed on the right side of the sixth through hole, the eighth through hole may be disposed to the left of the sixth through hole, and the ninth through hole may be disposed above the sixth through hole.

In addition, the grasping portion may include a first groove extending downward from an upper surface of the grasping portion, and the first groove may communicate with the sixth through hole and the ninth through hole.

In addition, a lower surface of the grasping portion in contact with the surgical instrument may protrude in a direction of the surgical instrument in a front area compared to a back area.

A forceps apparatus according to one aspect of the present disclosure for achieving the above object includes a first forceps unit and a second forceps unit connected to a motor respectively and facing each other, each of the first forceps unit and the second forceps unit include a base portion connected to a motor; a grasping portion disposed in front of the base portion and grasping a surgical instrument; a connecting portion connecting the base portion and the grasping portion; and a plurality of optical fibers, wherein some of the plurality of optical fibers are fixed to the grasping portion and others of the plurality of optical fibers are fixed to the base portion.

In this case, the plurality of optical fibers may include a first optical fiber disposed in a central region of the grasping portion and second to fifth optical fibers radially disposed with respect to the first optical fiber, and the first optical fiber may protrude further forward than the second to fifth optical fibers.

In addition, the two forceps units may be hinged at a portion connected to the motor, and a force measured by the two forceps units may satisfy the following Equation,

$$\\ T \cdot [ \begin{matrix}{F}_{xu} \\ {F}_{yu} \\ {F}_{zu} \\ {F}_{xl} \\ {F}_{yl} \\ {F}_{zl} \\ Δ {T}_{u} \\ Δ {T}_{l}\end{matrix} ] = [ \begin{matrix}{F}_{X} \\ {F}_{Y} \\ {F}_{Z} \\ {F}_{G} \\ {T}_{Z} \\ Δ T\end{matrix} ]$$

(where, T is

$$\\ [ \begin{matrix}0.5 & 0 & 0 & & 0 & 0 & 0 & 0 \\ 0 & cos ( θ ) & sin ( θ ) & 0 & - cos ( θ ) & - sin ( θ ) & 0 & 0 \\ 0 & - sin ( θ ) & cos ( θ ) & 0 & - sin ( θ ) & cos ( θ ) & 0 & 0 \\ 0 & 0.5 & 0.5 & 0 & 0.5 & 0 & 0 & 0 \\ 0.5 d & 0 & 0 & 0.5 d & 0 & 0 & 0 & 0 \\ 0 & 0 & 0 & 0 & 0 & 0 & 0.5 & 0.5\end{matrix} ] ,$$

Fₓᵤ is a force in the x-direction of the first forceps unit, F_{yu} is a force in the y-direction of the first forceps unit, F_{zu} is a force in the z-direction of the first forceps unit, Fₓₗ is a force in the x-direction of the second forceps unit, F_{yl} is a force in the y-direction of the second forceps unit, F_{zl} is a force in the z-direction of the second forceps unit, Tᵤ is a temperature of the first forceps unit, Tₗ is a temperature of the second forceps unit, F_{X} is a force in the x-direction of the forceps apparatus, F_{Y} is a force in the y-direction of the forceps apparatus, F_{Z} is a force in the z-direction of the forceps apparatus, T_{Z} is a torsion in the z-direction torsion of the forceps apparatus, T is a temperature of the forceps apparatus).

Through the present disclosure, it is possible to provide the forceps unit and the forceps apparatus capable of precisely measuring the 5-axis forces by compensating for the effect of temperature change.

### BRIEF DESCRIPTION OF THE DRAWINGS

FIG. 1 is a perspective view of a surgical apparatus according to an embodiment of the present disclosure.

FIG. 2 is an exploded perspective view of a surgical apparatus according to an embodiment of the present disclosure.

FIG. 3 is a perspective view of a forceps apparatus according to an embodiment of the present disclosure.

FIG. 4 is a perspective view of a forceps unit according to an embodiment of the present disclosure.

FIG. 5 is an exploded perspective view of a forceps unit according to an embodiment of the present disclosure.

FIG. 6 is a perspective view of a base portion according to an embodiment of the present disclosure.

FIG. 7 is a front view of a base portion according to an embodiment of the present disclosure.

FIG. 8 is a plan view of a base portion according to an embodiment of the present disclosure.

FIG. 9 is a bottom view of a base portion according to an embodiment of the present disclosure.

FIG. 10 is a perspective view of a connecting portion according to an embodiment of the present disclosure.

FIG. 11 is a front view of a connecting portion according to an embodiment of the present disclosure.

FIG. 12 is a plan view of a connecting portion according to an embodiment of the present disclosure.

FIG. 13 is a bottom view of a connecting portion according to an embodiment of the present disclosure.

FIG. 14 is a perspective view of a grasping portion according to an embodiment of the present disclosure.

FIG. 15 is a back view of a grasping portion according to an embodiment of the present disclosure.

FIG. 16 is a plan view of a grasping portion according to an embodiment of the present disclosure.

FIG. 17 is a bottom view of a grasping portion according to an embodiment of the present disclosure.

FIG. 18 is a perspective view of an optical fiber according to an embodiment of the present disclosure.

FIG. 19 is a right view of a forceps unit according to an embodiment of the present disclosure.

FIG. 20 is a B-B cross-sectional view of FIG. 19.

FIG. 21 is a A-A cross-sectional view of FIG. 19.

FIG. 22 is Equation 1 for measuring a force applied to a forceps unit.

FIG. 23 is a diagram showing how to obtain a force applied to a forceps apparatus from a force applied to a forceps unit.

FIG. 24 is Equation 2 for calculating a force applied to a forceps apparatus.

### DETAILED DESCRIPTION OF THE EMBODIMENTS

Hereinafter, embodiments disclosed in the present disclosure will be described in detail with reference to the accompanying drawings, however, regardless of the reference numerals, the same or similar components will be given the same reference numerals and redundant description thereof will be omitted.

In describing the embodiments disclosed in the present disclosure, when a component is referred to as being “connected” or “accessed” to other component, it may be directly connected or accessed to the other component, however, it may be understood that other components may be present in the middle.

In addition, in describing the embodiments disclosed in the present disclosure, when it is determined that the detailed description of the related known technology may obscure the subject matter of the embodiments disclosed in the present disclosure, the detailed description thereof will be omitted. In addition, the accompanying drawings are only for easily understanding the embodiments disclosed in the present disclosure, the technical spirit disclosed in the present disclosure is not limited by the accompanying drawings, and it should be understood that the accompanying drawings include all changes, equivalents, and substitutes included in the spirit and scope of the present disclosure.

On the other hand, terms of disclosure may be replaced with terms such as document, specification, description.

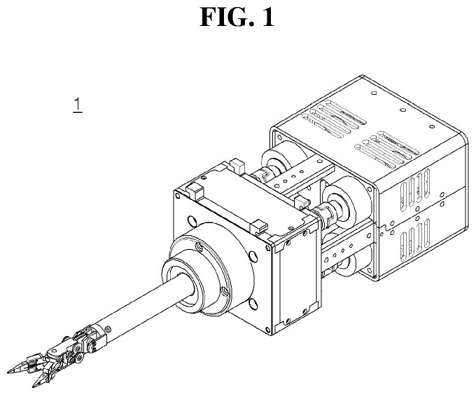

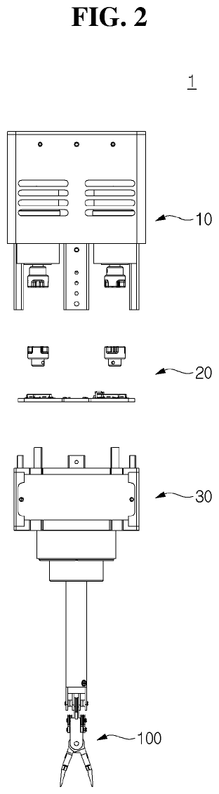

FIG. 1 is a perspective view of a surgical apparatus according to an embodiment of the present disclosure. FIG. 2 is an exploded perspective view of a surgical apparatus according to an embodiment of the present disclosure.

Referring to FIGS. 1 and 2, a surgical apparatus 1 according to an embodiment of the present disclosure may include a motor 10, a controller 20, a connection apparatus 30, and a forceps apparatus 100, but may be implemented except for some of these configurations, and does not exclude other additional configurations.

The motor 10 may provide a driving force. The motor 10 may be electrically connected to the controller 20. The motor 10 may be controlled by the controller 20. The motor 10 may provide the driving force to the forceps apparatus 100 through the connection apparatus 30.

The controller 20 may be electrically connected to the motor 10. The controller 20 may detect movements of the connection apparatus 30 and the forceps apparatus 100 and control driving of the forceps apparatus 100 using the motor 10.

The connection apparatus 30 may operate the forceps apparatus 100 by receiving the driving force from the motor 10. The connection apparatus 30 may be composed of wires and polys, but is not limited thereto and may be variously changed. The connection apparatus 30 may be detachably coupled to the motor 10 and the controller 20.

The forceps apparatus 100 may be connected to the connection apparatus 30. The forceps apparatus 100 may be electrically connected to the controller 20. The forceps apparatus 100 may be operated by receiving the driving force from the motor 10 under the control of the controller 20. The forceps apparatus 100 may grasp surgical instruments. The forceps apparatus 100 may be detachably coupled to a connecting portion 120. Through this, the forceps apparatus 100 can be separately separated and sterilized using a separate sterilizer.

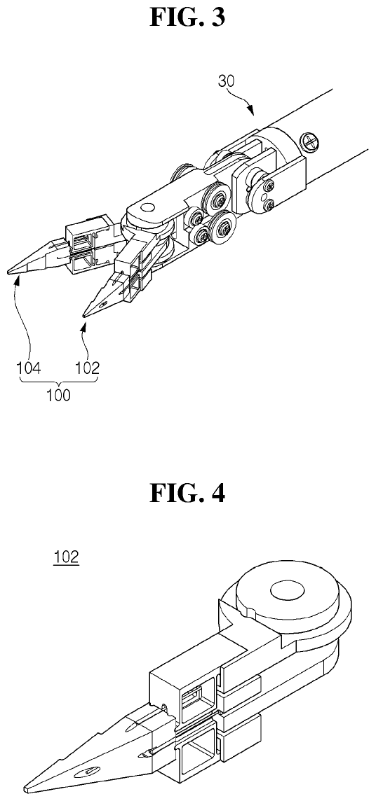

FIG. 3 is a perspective view of a forceps apparatus according to an embodiment of the present disclosure.

Referring to FIG. 3, the forceps apparatus 100 may include a plurality of forceps units 102 and 104. For example, forceps apparatus 100 may include two forceps units 102 and 104. The two forceps units 102 and 104 may be formed in symmetrical shapes. The two forceps units 102 and 104 may face each other. The two forceps units 102 and 104 may be hinged to the connection apparatus 30 connected to the motor 10. When front areas of the two forceps units 102 and 104 that are spaced apart from each other come closer to each other, the surgical instrument can be grasped. In one embodiment of the present disclosure, the controller 20 and the connection apparatus 30 may be understood as one component of the motor 10.

The two forceps units 102 and 104 may include a right forceps unit 102 and a left forceps unit 104.

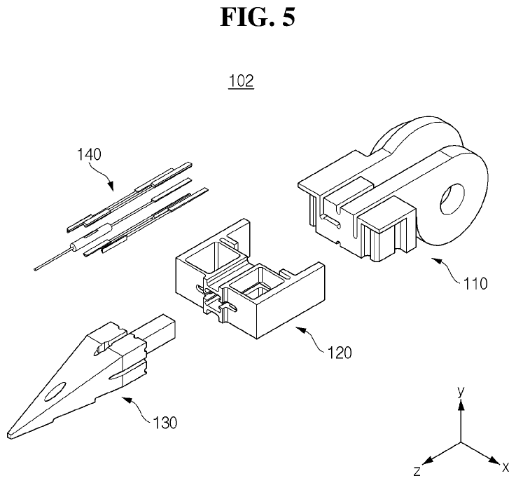

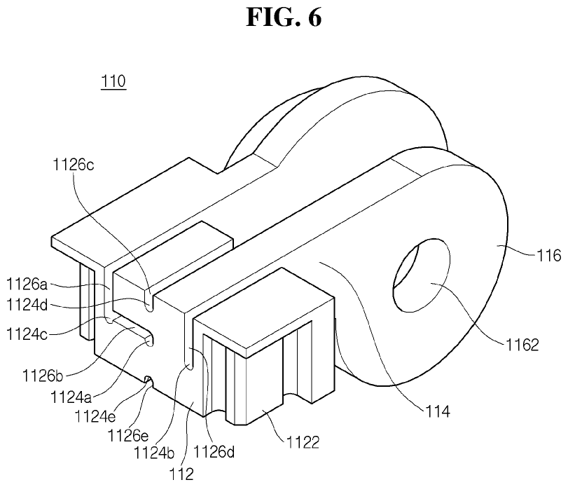

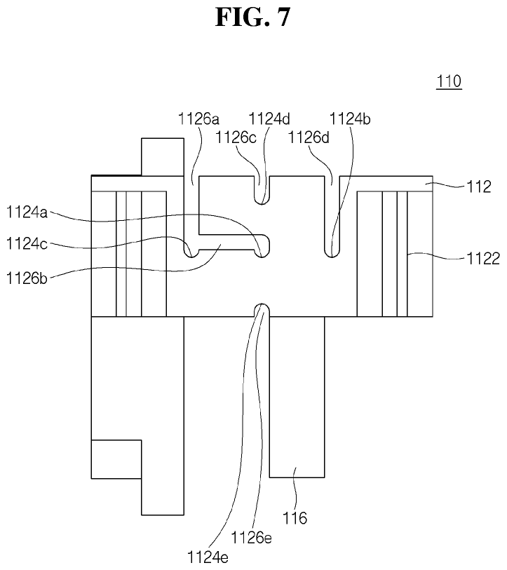

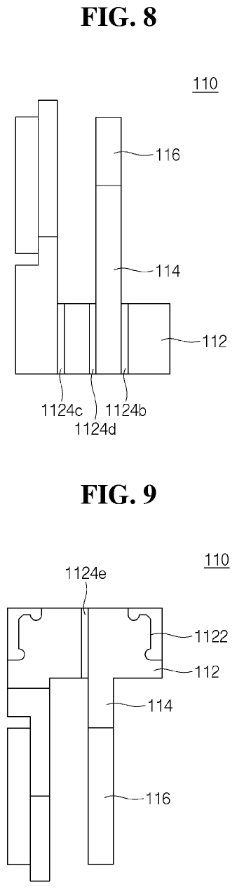

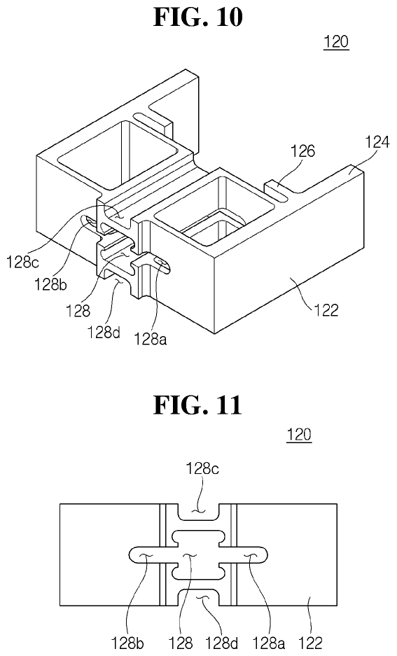

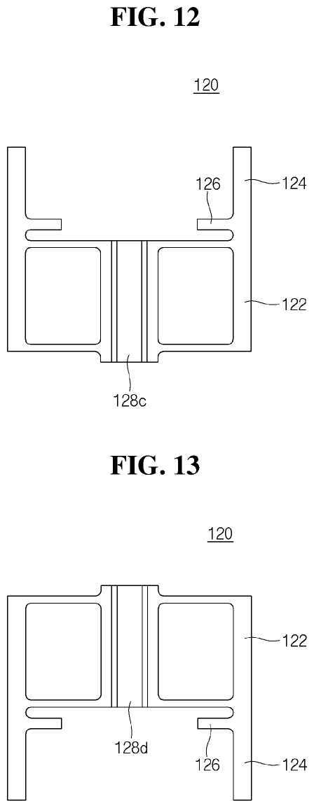

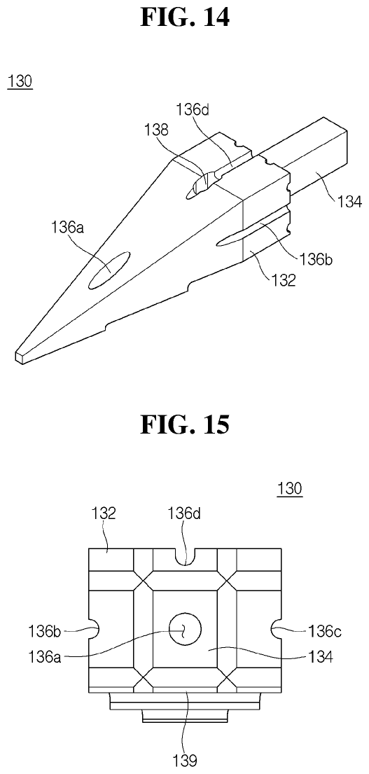

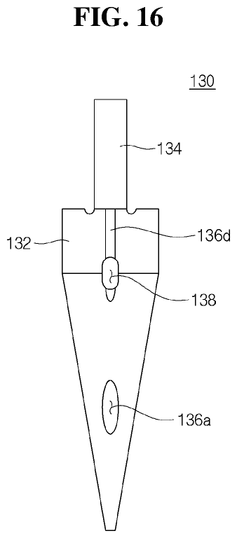

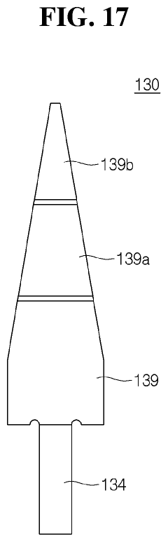

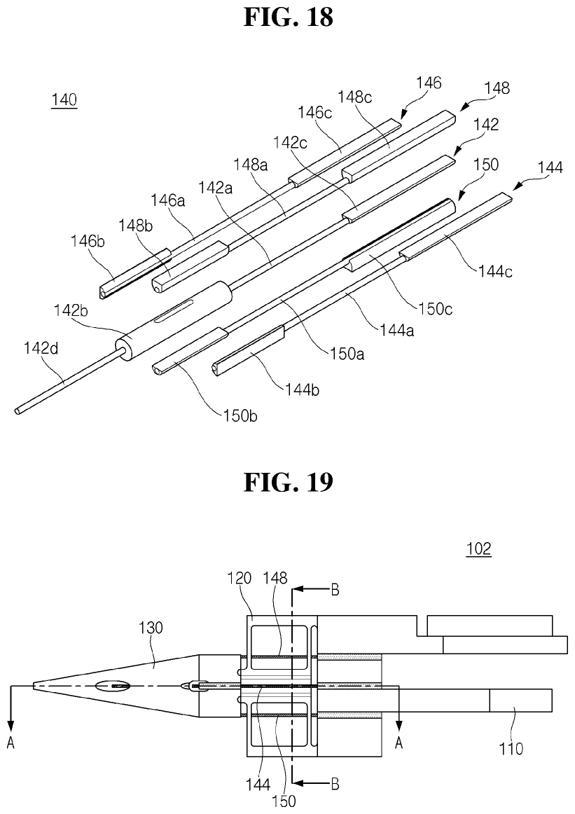

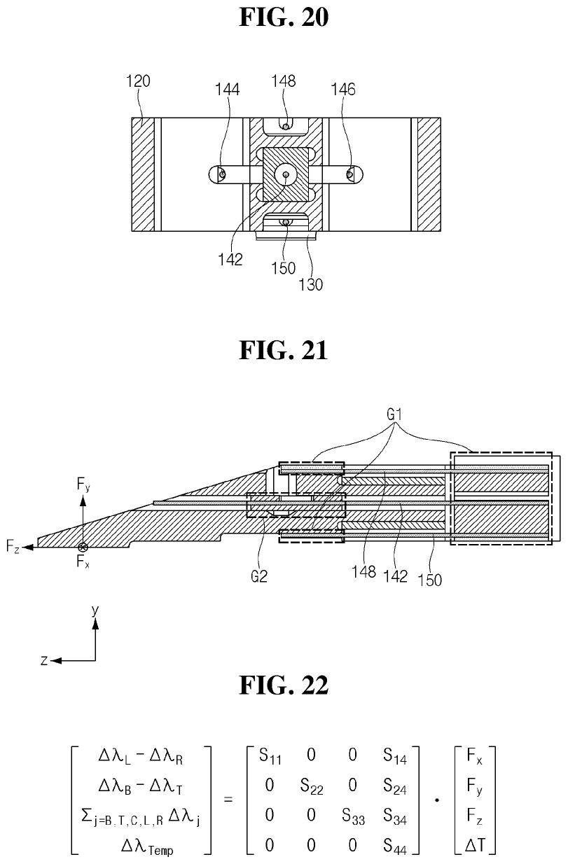

FIG. 4 is a perspective view of a forceps unit according to an embodiment of the present disclosure. FIG. 5 is an exploded perspective view of a forceps unit according to an embodiment of the present disclosure. FIG. 6 is a perspective view of a base portion according to an embodiment of the present disclosure. FIG. 7 is a front view of a base portion according to an embodiment of the present disclosure. FIG. 8 is a plan view of a base portion according to an embodiment of the present disclosure. FIG. 9 is a bottom view of a base portion according to an embodiment of the present disclosure. FIG. 10 is a perspective view of a connecting portion according to an embodiment of the present disclosure. FIG. 11 is a front view of a connecting portion according to an embodiment of the present disclosure. FIG. 12 is a plan view of a connecting portion according to an embodiment of the present disclosure. FIG. 13 is a bottom view of a connecting portion according to an embodiment of the present disclosure. FIG. 14 is a perspective view of a grasping portion according to an embodiment of the present disclosure. FIG. 15 is a back view of a grasping portion according to an embodiment of the present disclosure. FIG. 16 is a plan view of a grasping portion according to an embodiment of the present disclosure. FIG. 17 is a bottom view of a grasping portion according to an embodiment of the present disclosure. FIG. 18 is a perspective view of an optical fiber according to an embodiment of the present disclosure. FIG. 19 is a right view of a forceps unit according to an embodiment of the present disclosure. FIG. 20 is a B-B cross-sectional view of FIG. 19. FIG. 21 is a A-A cross-sectional view of FIG. 19.

Referring to FIGS. 4 to 21, the right forceps unit 102 may include a base portion 110, a connecting portion 120, a grasping portion 130, and an optical fiber 140, but may be implemented except for some of these configurations, and does not exclude other additional configurations. The left forceps unit 104 may be understood as the same configuration as the right forceps unit 102. The detailed configuration of the left forceps unit 104 may have a symmetrical arrangement with the detailed configuration of the right forceps unit 102.

The base portion 110 may be coupled to the connection apparatus 30 connected to the motor 10. The base portion 110 may be hinged to the connection apparatus 30. Alternatively, the base portion 110 may be directly coupled to the motor 10.

The base portion 110 may include a base body 112. The base body 112 may be coupled to the connecting portion 120. An adhesive may be interposed between the base body 112 and the connecting portion 120. The optical fiber 140 may be coupled to the base body 112. The base body 112 may be formed in a hexagonal shape as a whole.

The base body 112 may include first to fifth through holes 1124a, 1124b, 1124c, 1124d, and 1124e. The first to fifth through holes 1124a, 1124b, 1124c, 1124d, and 1124e may extend in the z-axis direction. The first to fifth through holes 1124a, 1124b, 1124c, 1124d, and 1124e may extend in a horizontal direction. The first to fifth through holes 1124a, 1124b, 1124c, 1124d, and 1124e may penetrate the base body 112. The first to fifth through holes 1124a, 1124b, 1124c, 1124d, and 1124e may be spaced apart from each other. The first to fifth through holes 1124a, 1124b, 1124c, 1124d, and 1124e may be respectively penetrated by first to fifth optical fibers 142, 144, 146, 148, and 150.

The first through hole 1124a may be penetrated by the first optical fiber 142. The first through hole 1124a may be formed in a central region of the base body 112. A second fixing portion 142c of the first optical fiber 142 may be fixed to the first through hole 1124a. An adhesive may be interposed between the first through hole 1124a and the second fixing portion 142c of the first optical fiber 142.

The first through hole 1124a may be connected to the third through hole 1124c. The first through hole 1124a may be connected to the third through hole 1124c in the horizontal direction. The first through hole 1124a may extend in the horizontal direction with the third through hole 1124c through a first connection hole 1126b. Alternatively, the first through hole 1124a may be connected to the second through hole 1124b in the horizontal direction. Through this, ease of manufacture of the first through hole 1124a may be improved.

The second through hole 1124b may be disposed on the right side of the first through hole 1124a. The second through hole 1124b may be penetrated by the second optical fiber 144. A fourth fixing portion 144c of the second optical fiber 144 may be fixed to the second through hole 1124b. An adhesive may be interposed between the second through hole 1124b and the fourth fixing portion 144c of the second optical fiber 144. The second through hole 1124b may extend downward from an upper surface of the base body 112. The second through hole 1124b may be connected to the upper surface of the base body 112 through a second connection hole 1126d.

The third through hole 1124c may be disposed on the left side of the first through hole 1124a. The third through hole 1124c may be penetrated by the third optical fiber 146. A sixth fixing portion 146c of the third optical fiber 146 may be fixed to the third through hole 1124c. An adhesive may be interposed between the third through hole 1124c and the sixth fixing portion 146c of the third optical fiber 146. The third through hole 1124c may extend downward from the upper surface of the base body 112. The third through hole 1124c may be connected to the upper surface of the base body 112 through a third connection hole 1126a.

The fourth through hole 1124d may be disposed above the first through hole 1124a. The fourth through hole 1124d may be penetrated by the fourth optical fiber 148. An eighth fixing portion 148c of the fourth optical fiber 148 may be fixed to the fourth through hole 1124d. An adhesive may be interposed between the fourth through hole 1124d and the eighth fixing portion 148c of the fourth optical fiber 148. The fourth through hole 1124d may extend downward from the upper surface of the base body 112. The fourth through hole 1124d may be connected to the upper surface of the base body 112 through a fourth connection hole 1126c.

The fifth through hole 1124e may be disposed below the first through hole 1124a. The fifth through hole 1124e may be penetrated by the fifth optical fiber 150. A tenth fixing portion 150c of the fifth optical fiber 150 may be fixed to the fifth through hole 1124e. An adhesive may be interposed between the fifth through hole 1124e and the tenth fixing portion 150c of the fifth optical fiber 150. The fifth through hole 1124e may extend upward from a lower surface of the base body 112. The fifth through hole 1124e may be connected to the lower surface of the base body 112 through a fifth connection hole 1126e.

The base body 112 may include a base connection groove 1122 extending from the front to the back. A first connection protrusion 124 of the connecting portion 120 may be disposed in the base connection groove 1122.

The base portion 110 may include a base connection portion 114. The base connection portion 114 may connect the base body 112 and a base coupling portion 116. The base connection portion 114 may extend backward from a back surface of the base body 112. The base connection portion 114 may include two base connection units spaced apart from each other.

The base portion 110 may include the base coupling portion 116. The base coupling portion 116 may be coupled to the motor 10 or the connection apparatus 30. Specifically, a hinge hole 1162 of the base coupling portion 116 may be hinged to the motor 10 or the connection apparatus 30. The base coupling portion 116 may include two base coupling units spaced apart from each other. Each of the base coupling units may be connected to each of the base coupling units.

The connecting portion 120 may be disposed between the base portion 110 and the grasping portion 130. The connecting portion 120 may connect the base portion 110 and the grasping portion 130. The connecting portion 120 may be penetrated by the optical fiber 140.

The connecting portion 120 may include a connecting body 122. The connecting body 122 may be penetrated by the optical fiber 140. The connecting body 122 may be coupled to the grasping portion 130.

The connecting body 122 may include a grasping hole 128. The grasping hole 128 may be formed in a central region of the connecting body 122. The grasping hole 128 may extend in the z-axis direction. A grasping protrusion 134 of the grasping portion 130 may be disposed in the grasping hole 128. An adhesive may be interposed between the grasping hole 128 and the grasping protrusion 134. The grasping hole 128 may be penetrated by the first optical fiber 142. A first brag 142a of the first optical fiber 142 may be disposed in the grasping hole 128.

The connecting body 122 may include a first fiber hole 128a. The first fiber hole 128a may be connected to the grasping hole 128. The first fiber hole 128a may be disposed on the right side of the grasping hole 128. The first fiber hole 128a may extend in the z-axis direction. The first fiber hole 128a may be penetrated by the second optical fiber 144. A third brag 144a of the second optical fiber 144 may be disposed in the first fiber hole 128a.

The connecting body 122 may include a second fiber hole 128b. The second fiber hole 128b may be connected to the grasping hole 128. The second fiber hole 128b may be disposed on the left side of the grasping hole 128. The second fiber hole 128b may extend in the z-axis direction. The second fiber hole 128b may be penetrated by the third optical fiber 146. A fourth brag 146a of the third optical fiber 146 may be disposed in the second fiber hole 128b.

The connecting body 122 may include a third fiber hole 128c. The third fiber hole 128c may be disposed above the grasping hole 128. The third fiber hole 128c may extend in the z-axis direction. The third fiber hole 128c may be penetrated by the fourth optical fiber 148. A fifth brag 148a of the fourth optical fiber 148 may be disposed in the third fiber hole 128c.

The connecting body 122 may include a fourth fiber hole 128d. The fourth fiber hole 128d may be disposed below the grasping hole 128. The fourth fiber hole 128d may extend in the z-axis direction. The fourth fiber hole 128d may be penetrated by the fifth optical fiber 150. A sixth brag 150a of the fifth optical fiber 150 may be disposed in the fourth fiber hole 128d.

The connecting portion 120 may include the first connection protrusion 124. The first connection protrusion 124 may extend backward from the back surface of the connecting body 122. The first connection protrusion 124 may be disposed in the base connection groove 1122 of the base body 112. An adhesive may be interposed between the first connection protrusion 124 and the base connection groove 1122.

The connection portion 120 may include a second connection protrusion 126. The second connection protrusion 126 may extend toward the z-axis from the first connection protrusion 124. The second connection protrusion 126 may be disposed on a front surface of the base body 112. An adhesive may be interposed between the second connection protrusion 126 and the front surface of the base body 112.

The grasping portion 130 may be disposed in front of the base portion 110. The grasping portion 130 may be coupled to the connecting portion 120. The optical fiber 140 may be fixed to the grasping portion 130. The grasping portion 130 may grasp surgical instruments.

The grasping portion 130 may include a grasping body 132. The grasping body 132 may be spaced apart from the connecting portion 120. The front area of the grasping body 132 may be formed in a cone shape. The front area of the grasping body 132 may be formed in a shape in which a cross-sectional area decreases toward the front. The back area of the grasping body 132 may be formed in a hexahedral shape. The first to fifth optical fibers 142, 144, 146, 148, and 150 may be fixed to the grasping body 132.

The grasping portion 130 may include the grasping protrusion 134. The grasping protrusion 134 may protrude backward from a central region of the back surface of the grasping body 132. The grasping protrusion 134 may be formed in the shape of a square pillar with an open center. The grasping protrusion 134 may extend in the z-axis direction. The grasping protrusion 134 may be coupled to the connecting portion 120. The grasping protrusion 134 may be disposed in the grasping hole 128 of the connecting portion 120. An adhesive may be interposed between the grasping protrusion 134 and the grasping hole 128 of the connecting portion 120.

The grasping portion 130 may include sixth through ninth through holes 136a, 136b, 136c, and 136d. The sixth to ninth through holes 136a, 136b, 136c, and 136d may be spaced apart from each other. The sixth to ninth through holes 136a, 136b, 136c, and 136d may be penetrated by the first to fourth optical fibers 142, 144, 146, and 148.

The sixth through hole 136a may penetrate central areas of the grasping body 132 and the grasping protrusion 134. The sixth through hole 136a may extend in the z-axis direction. The sixth through hole 136a may be penetrated by the first optical fiber 142. A first fixing portion 142b and a second brag 142d of the first optical fiber 142 may be disposed in the sixth through hole 136a. An adhesive may be interposed between the sixth through hole 136a and the first fixing portion 142b.

The seventh through hole 136b may be disposed on the right side of the sixth through hole 136a. The seventh through hole 136b may extend in the z-axis direction. The second optical fiber 144 may be disposed in the seventh through hole 136b. A third fixing portion 144b of the second optical fiber 144 may be disposed in the seventh through hole 136b. An adhesive may be interposed between the seventh through hole 136b and the third fixing portion 144b of the second optical fiber 144. The seventh through hole 136b may be formed in a rib shape connected to the right side of the grasping body 132 to improve space efficiency. Alternatively, the seventh through hole 136b may be spaced apart from the right side of the grasping body 132 and formed inside the grasping body 132.

The eighth through hole 136c may be disposed on the left side of the sixth through hole 136a. The eighth through hole 136c may extend in the z-axis direction. The third optical fiber 146 may be disposed in the eighth through hole 136c. A fifth fixing portion 146b of the third optical fiber 146 may be disposed in the eighth through hole 136c. An adhesive may be interposed between the eighth through hole 136c and the fifth fixing portion 146b of the third optical fiber 146. The eighth through hole 136c may be formed in a rib shape connected to the left side of the grasping body 132 to improve space efficiency. Alternatively, the eighth through hole 136c may be spaced apart from the left side of the grasping body 132 and formed inside the grasping body 132.

The ninth through hole 136d may be disposed above the sixth through hole 136a. The ninth through hole 136d may extend in the z-axis direction. The fourth optical fiber 148 may be disposed in the ninth through hole 136d. A seventh fixing portion 148b of the fourth optical fiber 148 may be disposed in the ninth through hole 136d. An adhesive may be interposed between the ninth through hole 136d and the seventh fixing portion 148b of the fourth optical fiber 148. The ninth through hole 136d may be formed in a rib shape connected to the upper surface of the grasping body 132 to improve space efficiency. Alternatively, the ninth through hole 136d may be spaced apart from the upper surface of the grasping body 132 and formed inside the grasping body 132.

The grasping portion 130 may include a first groove 138. The first groove 138 may extend downward from the upper surface of the grasping body 132. The first groove 138 may extend in the y-axis direction. The first groove 138 may be connected to the sixth through hole 136a. The first groove 138 may be connected to the ninth through hole 136d. An adhesive may be supplied between the sixth through hole 136a and the first fixing portion 142b through the first groove 138.

The fifth optical fiber 150 may be disposed on a lower surface 139 of the grasping portion 130. The fifth optical fiber 150 may be fixed to the lower surface 139 of the grasping portion 130. An adhesive may be interposed between the lower surface 139 of the grasping portion 130 and a ninth fixing portion 150b of the fifth optical fiber 150.

The lower surface 139 of the grasping portion 130 may protrude in a direction of the surgical instrument in the front area compared to the back area. Specifically, a central area 139a of the lower surface 139 of the grasping portion 130 may protrude downward compared to the back area, and a front area 139b may protrude downward compared to the central area 139a.

A part of the optical fiber 140 may be fixed to the grasping portion 130 and another part may be fixed to the base portion 110. The optical fiber 140 may measure the force applied to the right forceps unit 102 and compensate for the effect of temperature.

The optical fiber 140 may include a plurality of optical fibers 142, 144, 146, 148, and 150.

The plurality of optical fibers 142, 144, 146, 148, and 150 may include first to fifth optical fibers 142, 144, 146, 148, and 150.

The first optical fiber 142 may be disposed in the central area of the grasping portion 130. The first optical fiber 142 may protrude forward compared to the second to fifth optical fibers 144, 146, 148, and 150. Through this, since 5-axis forces may be measured by compensating for the effect of temperature change, the force applied by the surgical instruments may be accurately and precisely measured.

In one embodiment of the present disclosure, forward may be interpreted as meaning the z-axis direction, and backward may be interpreted as meaning the −z axis direction.

The first optical fiber 142 may include the first fixing portion 142b fixed to the grasping portion 130, the second fixing portion 142c fixed to the base portion 110, the first brag 142a disposed between the first fixing portion 142b and the second fixing portion 142c, and the second brag 142d disposed in front of the first fixing portion 142b. The first brag 142a may measure the z-axis force F_{z} through a change in length. The second brag 142d may measure the temperature change through the change in length.

The second to fifth optical fibers 144, 146, 148, and 150 may be radially disposed with respect to the first optical fiber 142.

The second optical fiber 144 may be disposed on the right side of the first optical fiber 142. The second optical fiber 144 may include the third fixing portion 144b fixed to the grasping portion 130, the fourth fixing portion 144c fixed to the base portion 110, and the third brag 144a disposed between the third fixing portion 144b and the fourth fixing portion 144c. The third brag 144a may measure the x-axis force Fₓ and the z-axis force F_{z} through the change in length.

The third optical fiber 146 may be disposed on the left side of the first optical fiber 142. The third optical fiber 146 may include the fifth fixing portion 146b fixed to the grasping portion 130, the sixth fixing portion 146c fixed to the base portion 110, and the fourth brag 146a disposed between the fifth fixing portion 146b and the sixth fixing portion 146c. The fourth brag 146a may measure the x-axis force Fₓ and the z-axis force F_{z} through the change in length.

The fourth optical fiber 148 may be disposed above the first optical fiber 142. The fourth optical fiber 148 may include the seventh fixing portion 148b fixed to the grasping portion 130, the eighth fixing portion 148c fixed to the base portion 110, and the fifth brag 148a disposed between the seventh fixing portion 148b and the eighth fixing portion 148c. The fifth brag 148a may measure the y-axis force F_{y} and the z-axis force F_{z} through the change in length.

The fifth optical fiber 150 may be disposed below the first optical fiber 142. The fifth optical fiber 150 may include the ninth fixing portion 150b fixed to the grasping portion 130, the tenth fixing portion 150c fixed to the base portion 110, and the sixth brag 150a disposed between the ninth fixing portion 150b and the tenth fixing portion 150c. The sixth brag 150a may measure the y-axis force F_{y} and the z-axis force F_{z} through the change in length.

The first brag 142a and the third to sixth brags 144a, 146a, 148a, and 150a may be disposed on the same plane. Through this, the 5-axis forces may be measured.

A back end of the first fixing portion 142b, a back end of the third fixing portion 144b, a back end of the fifth fixing portion 146b, a back end of the seventh fixing portion 148b, and a back end of the ninth fixing portion 150b may be disposed on the same plane. A front end of the second fixing portion 142c, a front end of the fourth fixing portion 144c, a front end of the sixth fixing portion 146c, a front end of the eighth fixing portion 148c, and a front end of the tenth fixing portion 150c may be disposed on the same plane. Through this, the 5-axis forces may be precisely measured.

The second optical fiber 144 may be disposed symmetrically with the third optical fiber 146 with respect to the first optical fiber 142. The fourth optical fiber 148 may be disposed symmetrically with the fifth optical fiber 150 with respect to the first optical fiber 142. Intervals between the second to fifth optical fibers 144, 146, 148, and 150 may be the same.

A front end of the first optical fiber 142 may be a free end. Specifically, the front end of the first optical fiber 142 may be in an unfixed state. Through this, it is possible to measure the change in length of the second brag 142d.

FIG. 22 is Equation 1 for measuring a force applied to a forceps unit.

Referring to FIG. 22, the first to sixth brags 142a, 142d, 144a, 146a, 148a, and 150a may satisfy the following Equation.

$$\\ \begin{matrix}[ \begin{matrix}Δ {λ}_{L} - Δ {λ}_{R} \\ Δ {λ}_{B} - Δ {λ}_{T} \\ {\sum }_{j = B , T , C , L , R} Δ {λ}_{j} \\ Δ {λ}_{Temp}\end{matrix} ] = [ \begin{matrix}{S}_{11} & 0 & 0 & {S}_{14} \\ 0 & {S}_{22} & 0 & {S}_{24} \\ 0 & 0 & {S}_{33} & {S}_{34} \\ 0 & 0 & 0 & {S}_{44}\end{matrix} ] \cdot [ \begin{matrix}{F}_{x} \\ {F}_{y} \\ {F}_{z} \\ Δ T\end{matrix} ] & [ Equation 1 ]\end{matrix}$$

Where, λ_{C} may mean a change in length of the first brag 142a, λ_{Temp} may mean a change in length of the second brag 142d, λ_{R} may mean a change in length of the third brag 144a, λ_{L} may mean a change in length of the fourth brag 146a, λ_{T} may mean a change in length of the fifth brag 148a, λ_{B} may mean a change in length of the sixth brag 150a.

In addition, S₁₁, S₁₄, S₂₂, S₂₄, S₃₃, S₃₄, and S₄₄ are constants and may be determined according to physical property values of the first to sixth brags 142a, 142d, 144a, 146a, 148a, and 150a. In the middle matrix, 0 may be interpreted as meaning a negligibly small value compared to other constants.

Through this, the x-axis force Fₓ, the y-axis force F_{y}, the z-axis force F_{z}, and the temperature change ΔT applied to the forceps units 102 and 104 can be obtained.

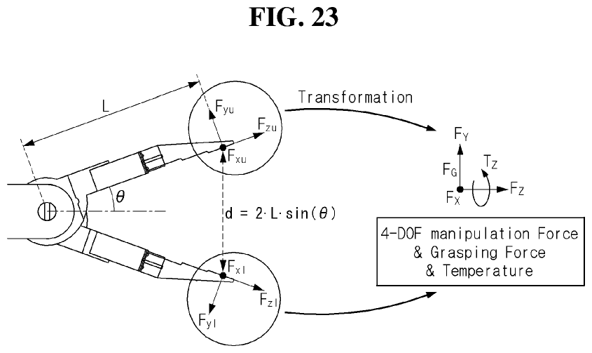

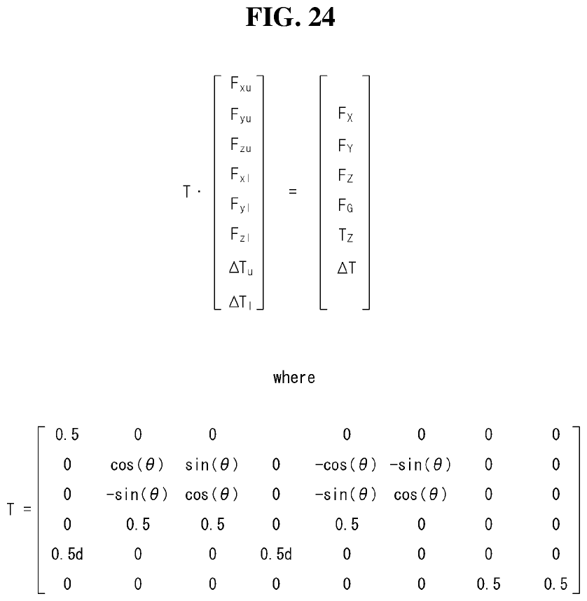

FIG. 23 is a diagram showing how to obtain a force applied to a forceps apparatus from a force applied to a forceps unit. FIG. 24 is Equation 2 for calculating a force applied to a forceps apparatus.

Referring to FIGS. 23 and 24, the 5-axis forces F_{X}, F_{Y}, F_{Z}, F_{G}, T_{Z} and temperature change ΔT measured by the two forceps units 102 and 104 may satisfy the following Equation.

$$\\ \begin{matrix}T \cdot [ \begin{matrix}{F}_{xu} \\ {F}_{yu} \\ {F}_{zu} \\ {F}_{xl} \\ {F}_{yl} \\ {F}_{zl} \\ Δ {T}_{u} \\ Δ {T}_{l}\end{matrix} ] = [ \begin{matrix}{F}_{X} \\ {F}_{Y} \\ {F}_{Z} \\ {F}_{G} \\ {T}_{Z} \\ Δ T\end{matrix} ] & [ Equation 2 ]\end{matrix}$$

Where, T is

$$\\ [ \begin{matrix}0.5 & 0 & 0 & & 0 & 0 & 0 & 0 \\ 0 & cos ( θ ) & sin ( θ ) & 0 & - cos ( θ ) & - sin ( θ ) & 0 & 0 \\ 0 & - sin ( θ ) & cos ( θ ) & 0 & - sin ( θ ) & cos ( θ ) & 0 & 0 \\ 0 & 0.5 & 0.5 & 0 & 0.5 & 0 & 0 & 0 \\ 0.5 d & 0 & 0 & 0.5 d & 0 & 0 & 0 & 0 \\ 0 & 0 & 0 & 0 & 0 & 0 & 0.5 & 0.5\end{matrix} ] ,$$

Fₓᵤ may mean a force in the x-direction of the first forceps unit, F_{yu} may mean a force in the y-direction of the first forceps unit, F_{zu} may mean a force in the z-direction of the first forceps unit, Fₓₗ may mean a force in the x-direction of the second forceps unit, F_{yl} may mean a force in the y-direction of the second forceps unit, F_{zl} may mean a force in the z-direction of the second forceps unit, Tᵤ may mean a temperature of the first forceps unit, Tₗ may mean a temperature of the second forceps unit, F_{X} may mean a force in the x-direction of the forceps apparatus, F_{Y} may mean a force in the y-direction of the forceps apparatus, F_{Z} may mean a force in the z-direction of the forceps apparatus, T_{Z} may mean a torsion in the z-direction torsion of the forceps apparatus, T may mean a temperature of the forceps apparatus.

The first matrix, the T matrix, may mean physical property values of the forceps apparatus 100 in a state in which a surgical instrument is grasped. Here, L may mean a distance between a position where the surgical instrument is grasped and a central area of the hinge hole 1162, θ may mean half of the angle between the right forceps apparatus 102 and the left forceps apparatus 104, and d may mean 2*L*sinθ.

The second matrix may mean the 3-axis forces and temperature change measured by the right forceps unit 102, and the 3-axis forces and temperature change measured by the left forceps unit 104. This may be obtained through Equation 1 described above, respectively.

The third matrix may mean the 5-axis forces F_{X}, F_{Y}, F_{Z}, F_{G}, T_{Z} and temperature change ΔT of the forceps apparatus 100.

That is, through the forceps apparatus 100 according to an embodiment of the present disclosure, since the 5-axis forces F_{X}, F_{Y}, F_{Z}, F_{G}, T_{Z} generated when grasping the surgical instrument can be obtained by compensating the error due to the temperature change ΔT, it is possible to improve precision and reliability.

Some or other embodiments of the present disclosure described above are not exclusive or distinct from one another. Some or other embodiments of the present disclosure described above may be used in combination or combined with each configuration or function.

For example, it means that configuration A described in specific embodiments and/or drawings and configuration B described in other embodiments and/or drawings may be combined. In other words, even when the combination between the components is not described directly, it means that the combination is possible except when it is described as not possible to combine.

The above detailed description should not be construed as limiting in all respects and should be considered as illustrative. The scope of the present disclosure should be determined by reasonable interpretation of the appended claims, and all changes within the equivalent scope of the present disclosure are included in the scope of the present disclosure.

What is claimed is:

1. A forceps unit comprising:
a base portion connected to a motor;
a grasping portion disposed in front of the base portion and grasping a surgical instrument;
a connecting portion connecting the base portion and the grasping portion; and
a plurality of optical fibers, wherein some of the plurality of optical fibers are fixed to the grasping portion and others of the plurality of optical fibers are fixed to the base portion,
wherein the plurality of optical fibers include a first optical fiber disposed in a central region of the grasping portion and second to fifth optical fibers radially disposed with respect to the first optical fiber, and
the first optical fiber protrudes further forward than the second to fifth optical fibers.

2. The forceps unit of claim 1, wherein the first optical fiber includes a first fixing portion fixed to the grasping portion, a second fixing portion fixed to the base portion, a first brag disposed between the first fixing portion and the second fixing portion, and a second brag disposed in front of the first fixing portion.

3. The forceps unit of claim 2, wherein the second optical fiber includes a third fixing portion fixed to the grasping portion, a fourth fixing portion fixed to the base portion, and a third brag disposed between the third fixing portion and the fourth fixing portion,
the third optical fiber includes a fifth fixing portion fixed to the grasping portion, a sixth fixing portion fixed to the base portion, and a fourth brag disposed between the fifth fixing portion and the sixth fixing portion,
the fourth optical fiber includes a seventh fixing portion fixed to the grasping portion, an eighth fixing portion fixed to the base portion, and a fifth brag disposed between the seventh fixing portion and the eighth fixing portion, and
the fifth optical fiber includes a ninth fixing portion fixed to the grasping portion, a tenth fixing portion fixed to the base portion, and a sixth brag disposed between the ninth fixing portion and the tenth fixing portion.

4. The forceps unit of claim 3, wherein the first brag and the third to sixth brags are disposed on the same plane.

5. The forceps unit of claim 3, wherein a back end of the first fixing portion, a back end of the third fixing portion, a back end of the fifth fixing portion, a back end of the seventh fixing portion, and a back end of the ninth fixing portion are disposed on the same plane, and
a front end of the second fixing portion, a front end of the fourth fixing portion, a front end of the sixth fixing portion, a front end of the eighth fixing portion, and a front end of the tenth fixing portion are disposed on the same plane.

6. The forceps unit of claim 3, wherein the first to sixth brags satisfy the following Equation,
$$\\ [ \begin{matrix}Δ {λ}_{L} - Δ {λ}_{R} \\ Δ {λ}_{B} - Δ {λ}_{T} \\ {\sum }_{j = B , T , C , L , R} Δ {λ}_{j} \\ Δ {λ}_{Temp}\end{matrix} ] = [ \begin{matrix}{S}_{11} & 0 & 0 & {S}_{14} \\ 0 & {S}_{22} & 0 & {S}_{24} \\ 0 & 0 & {S}_{33} & {S}_{34} \\ 0 & 0 & 0 & {S}_{44}\end{matrix} ] \cdot [ \begin{matrix}{F}_{x} \\ {F}_{y} \\ {F}_{z} \\ Δ T\end{matrix} ]$$
(where, λ_{C} is a change in length of the first brag, λ_{Temp} is a change in length of the second brag, λ_{R} is a change in length of the third brag, λ_{L} is a change in length of the fourth brag, λ_{T} is a change in length of the fifth brag, λ_{B} is a change in length of the sixth brag).

7. The forceps unit of claim 1, wherein the second optical fiber is disposed symmetrically with the third optical fiber with respect to the first optical fiber,
the fourth optical fiber is disposed symmetrically with the fifth optical fiber with respect to the first optical fiber, and
intervals between the second to fifth optical fibers are the same.

8. The forceps unit of claim 1, wherein the front end of the first optical fiber is a free end.

9. The forceps unit of claim 1, wherein the base portion includes first to fifth through holes respectively penetrated by the first to fifth optical fibers and extending in a horizontal direction,
the first through hole is penetrated by the first optical fiber,
the second through hole is disposed on the right side of the first through hole,
the third through hole is disposed on the left side of the first through hole,
the fourth through hole is disposed above the first through hole,
the fifth through hole is disposed below the first through hole,
the second to fourth through holes extend downward from an upper surface of the base portion, and
the first through hole is connected to the second through hole or the third through hole.

10. The forceps unit of claim 1, wherein the grasping portion includes sixth to ninth through holes respectively penetrated by the first to fourth optical fibers,
the sixth through hole is penetrated by the first optical fiber,
the seventh through hole is disposed on the right side of the sixth through hole,
the eighth through hole is disposed to the left of the sixth through hole, and
the ninth through hole is disposed above the sixth through hole.

11. The forceps unit of claim 10, wherein the grasping portion includes a first groove extending downward from an upper surface of the grasping portion, and
the first groove communicates with the sixth through hole and the ninth through hole.

12. The forceps unit of claim 1, wherein a lower surface of the grasping portion in contact with the surgical instrument protrudes in a direction of the surgical instrument in a front area compared to a back area.

13. A forceps apparatus comprising:
a first forceps unit and a second forceps unit connected to a motor respectively and facing each other,
each of the first forceps unit and the second forceps unit include:
a base portion connected to a motor;
a grasping portion disposed in front of the base portion and grasping a surgical instrument;
a connecting portion connecting the base portion and the grasping portion; and
a plurality of optical fibers, wherein some of the plurality of optical fibers are fixed to the grasping portion and others of the plurality of optical fibers are fixed to the base portion,
wherein the plurality of optical fibers include a first optical fiber disposed in a central region of the grasping portion and second to fifth optical fibers radially disposed with respect to the first optical fiber, and
the first optical fiber protrudes further forward than the second to fifth optical fibers.

14. The forceps apparatus of claim 13, wherein the two forceps units are hinged at a portion connected to the motor, and
a force measured by the two forceps units satisfies the following Equation,
$$\\ T \cdot [ \begin{matrix}{F}_{xu} \\ {F}_{yu} \\ {F}_{zu} \\ {F}_{xl} \\ {F}_{yl} \\ {F}_{zl} \\ Δ {T}_{u} \\ Δ {T}_{l}\end{matrix} ] = [ \begin{matrix}{F}_{X} \\ {F}_{Y} \\ {F}_{Z} \\ {F}_{G} \\ {T}_{Z} \\ Δ T\end{matrix} ]$$
(where, T is
$$\\ [ \begin{matrix}0.5 & 0 & 0 & & 0 & 0 & 0 & 0 \\ 0 & cos ( θ ) & sin ( θ ) & 0 & - cos ( θ ) & - sin ( θ ) & 0 & 0 \\ 0 & - sin ( θ ) & cos ( θ ) & 0 & - sin ( θ ) & cos ( θ ) & 0 & 0 \\ 0 & 0.5 & 0.5 & 0 & 0.5 & 0 & 0 & 0 \\ 0.5 d & 0 & 0 & 0.5 d & 0 & 0 & 0 & 0 \\ 0 & 0 & 0 & 0 & 0 & 0 & 0.5 & 0.5\end{matrix} ] ,$$
Fₓᵤ is a force in the x-direction of the first forceps unit, F_{yu} is a force in the y-direction of the first forceps unit, F_{zu} is a force in the z-direction of the first forceps unit, Fₓₗ is a force in the x-direction of the second forceps unit, F_{yl} is a force in the y-direction of the second forceps unit, F_{zl} is a force in the z-direction of the second forceps unit, Tᵤ is a temperature of the first forceps unit, Tₗ is a temperature of the second forceps unit, F_{X} is a force in the x-direction of the forceps apparatus, F_{Y} is a force in the y-direction of the forceps apparatus, F_{Z} is a force in the z-direction of the forceps apparatus, T_{Z} is a torsion in the z-direction torsion of the forceps apparatus, T is a temperature of the forceps apparatus).
